(19) United States
(12) Patent Application Publication
(10) Pub. No.: US 2026/0016442 A1
(43) Pub. Date: Jan. 15, 2026

(54) GAS SENSOR

(71) Applicant: NGK INSULATORS, LTD., Nagoya-shi (JP)

(72) Inventors: Atsuhiko MASUDA, Nagoya-shi (JP); Daichi ICHIKAWA, Nagoya-shi (JP)

(21) Appl. No.: 19/332,117

(22) Filed: Sep. 18, 2025

Related U.S. Application Data
(63) Continuation of application No. PCT/JP2024/006538, filed on Feb. 22, 2024.

(30) Foreign Application Priority Data
Mar. 30, 2023 (JP) 2023-055668

(51) Int. Cl. G01N 27/41; G01N 27/407; G01N 33/00

(52) U.S. Cl. CPC G01N 27/41; G01N 27/4071; G01N 33/0027

(57) ABSTRACT

A gas sensor includes a sensor element and a control device, and configured to measure a water concentration in a measurement gas, wherein the control device performs: a first pump cell control processing in which oxygen is pumped out from around a first inner electrode; a second pump cell control processing in which oxygen is pumped to around a second inner electrode by controlling the second pump cell; a third pump cell control processing in which oxygen is pumped to around a third inner electrode by controlling the third pump cell; and the control device derives the water concentration in the measurement gas based on the second pump current flowing through the second pump cell by the second pump cell control processing, while taking into account the third pump current flowing through the third pump cell by the third pump cell control processing.

### CROSS-REFERENCE TO RELATED APPLICATION

This application is a continuation application of PCT/JP2024/006538, filed on Feb. 22, 2024, which claims the benefit of priority of Japanese Application No. 2023-055668, filed on Mar. 30, 2023, the entire contents of which are incorporated herein by reference.

### BACKGROUND OF THE INVENTION

### 1. Field of the Invention

The present invention relates to a gas sensor.

### 2. Description of the Related Art

Hitherto, a gas sensor that detects the concentration of water in a measurement gas such as exhaust gas from an automobile have been known. For example, PTL 1 describes a gas sensor comprising a sensor element including an oxygen-ion-conductive solid electrolyte layer, which specifies the concentrations of water vapor components and carbon dioxide components in the measurement gas. In this gas sensor, the oxygen partial pressure in a first internal cavity of a sensor element is adjusted so that all of the water vapor component and the carbon dioxide component in the measurement-object gas are substantially decomposed in the first internal cavity. The gas sensor then supplies oxygen to a second internal cavity by a first measurement electrochemical pumping cell so that the hydrogen generated by decomposition of the water vapor component is selectively burned in the second internal cavity, and identifies the concentration of the water vapor component present in the measurement-object gas based on the magnitude of a current flowing then. In addition, this gas sensor supplies oxygen to the surface of a second measurement inner electrode by a second measurement electrochemical pumping cell so that the carbon monoxide generated by decomposition of the carbon dioxide component is selectively burned on the surface of the second measurement inner electrode, and identifies the concentration of the carbon dioxide component present in the measurement-object gas based on the magnitude of a current flowing then.

### CITATION LIST

### Patent Literature

PTL 1: JP 5918177 B

### SUMMARY OF THE INVENTION

In such gas sensors, it has been found that even when the concentration of the water vapor component (water concentration) in the measurement gas is the same, the measurement result of the water concentration by the gas sensor may vary depending on the concentration of the carbon dioxide in the measurement gas. That is, the measurement accuracy of the water concentration may be degraded due to the presence of carbon dioxide in the measurement gas.

The present invention was made to solve such a problem, and its main object is to suppress a decrease in the measurement accuracy of the water concentration caused by carbon dioxide in the measurement gas.

The present invention employs the following configuration to achieve the above-described main object.

[1] A gas sensor according to the present invention is a gas sensor including a sensor element and a control device, and configured to measure a water concentration in a measurement gas, wherein: the sensor element includes: an element body having an oxygen-ion-conductive solid electrolyte layer and a measurement gas flow path formed therein for introducing and allowing flow of the measurement gas; a first pump cell including a first inner electrode disposed in a first chamber of the measurement gas flow path, and a first outer electrode disposed on the outside of the element body; a second pump cell including a second inner electrode disposed in a second chamber located downstream of the first chamber of the measurement gas flow path, and a second outer electrode disposed on the outside of the element body; a third pump cell including a third inner electrode disposed in a third chamber located downstream of the second chamber of the measurement gas flow path, and a third outer electrode disposed on the outside of the element body; the control device performs: a first pump cell control processing in which oxygen is pumped out from around the first inner electrode to around the first outer electrode by controlling the first pump cell, thereby reducing water and carbon dioxide in the measurement gas in the first chamber; a second pump cell control processing in which oxygen is pumped from around the second outer electrode to around the second inner electrode by controlling the second pump cell, thereby oxidizing hydrogen generated by the reduction of water in the first chamber in the second chamber; a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and the control device derives the water concentration in the measurement gas based on the second pump current flowing through the second pump cell by the second pump cell control processing, while taking into account the third pump current flowing through the third pump cell by the third pump cell control processing.

In this gas sensor, the water concentration in the measurement gas is derived based on the second pump current by taking into account the third pump current. Here, the second pump current flowing through the second pump cell by the second pump cell control processing correlates with the water concentration in the measurement gas. Further, the third pump current flowing through the third pump cell by the third pump cell control processing correlates with the carbon dioxide concentration in the measurement gas. However, the second pump current may vary depending on the carbon dioxide concentration in the measurement gas even when the water concentration in the measurement gas is the same. Therefore, by deriving the water concentration in the measurement gas based on the second pump current while taking into account the third pump current, which correlates with the carbon dioxide concentration in the measurement gas, it is possible to suppress a decrease in the measurement accuracy of the water concentration caused by carbon dioxide in the measurement gas.

[2] In the above gas sensor (the gas sensor described in [1]), the control device may derive the water concentration based on a corrected second pump current obtained by correcting the second pump current taking into account the third pump current, or derive the water concentration by correcting a provisional water concentration based on the second pump current taking into account the third pump current.

[3] In the above gas sensor (the gas sensor described in [2]), the control device may derive the corrected second pump current or the water concentration by applying a correction in such a manner that the greater the absolute value of the third pump current, the more the absolute value of the second pump current or the provisional water concentration tends to decrease. Here, even when the water concentration in the measurement gas is the same, the absolute value of the second pump current tends to increase as the carbon dioxide concentration in the measurement gas increases. This is considered to be because the higher the carbon dioxide concentration in the measurement gas, the greater the amount of carbon monoxide generated by the reduction of carbon dioxide in the first chamber, and a portion of the carbon monoxide is oxidized not in the third chamber but in the second chamber upstream of the third chamber. Therefore, by applying a correction in such a manner that the greater the absolute value of the third pump current, which correlates with the carbon dioxide concentration, the more the absolute value of the second pump current or the provisional water concentration tends to decrease, it is possible to more reliably suppress a decrease in the measurement accuracy of the water concentration caused by carbon dioxide in the measurement gas.

[4] In the above gas sensor (the gas sensor described in [2] or [3]), the control device may derive a correction value based on the absolute value of the third pump current, and correct the second pump current or the provisional water concentration using the correction value. In this case, the control device may derive the correction value based on both the absolute value of the second pump current and the absolute value of the third pump current, and correct the second pump current using the derived correction value. Alternatively, the control device may derive the correction value based on both the absolute value of the third pump current and the provisional water concentration, and correct the provisional water concentration using the derived correction value.

[5] In the above gas sensor (the gas sensor described in [4]), the correction value may be a subtraction value to the absolute value of the second pump current or the provisional water concentration, and the control device may correct the second pump current or the provisional water concentration by subtracting the correction value from the absolute value of the second pump current or the provisional water concentration.

[6] In the above gas sensor (the gas sensor described in [5]), the control device may correct the second pump current or the provisional water concentration using the same correction value derived based on the absolute value of the third pump current, regardless of the magnitude of the absolute value of the second pump current or the provisional water concentration. When the correction value is the subtraction value, the second pump current or the provisional water concentration can be corrected with relatively high accuracy even using the same correction value based on the absolute value of the third pump current, regardless of the magnitude of the absolute value of the second pump current or the provisional water concentration. Therefore, the correction can be performed more easily compared to the case where the correction value is derived based on not only the absolute value of the third pump current but also the absolute value of the second pump current or the provisional water concentration.

[7] In the above gas sensor (the gas sensor described in any one of [1] to [6]), the control device may derive a carbon dioxide concentration in the measurement gas based on the third pump current. In this manner, this gas sensor can measure not only the water concentration but also the carbon dioxide concentration. In this case, the control device may derive the water concentration based on the second pump current while taking into account the carbon dioxide concentration derived based on the absolute value of the third pump current. For example, a correction taking the carbon dioxide concentration into account may be applied to the second pump current, or a correction taking the carbon dioxide concentration into account may be applied to the provisional water concentration. These embodiments are also included as specific examples of deriving the water concentration based on the second pump current while taking into account the third pump current. For example, the control device may derive the correction value based on the carbon dioxide concentration derived from the absolute value of the third pump current.

[8] In the above gas sensor (the gas sensor described in any one of [1] to [7]), at least two of the first outer electrode, the second outer electrode, and the third outer electrode may be shared electrodes.

[9] In the above gas sensor (the gas sensor described in any one of [1] to [8]), the sensor element may include a reference electrode disposed inside the element body to contact a reference gas, and the control device may control the first pump cell such that a first voltage between the reference electrode and the first inner electrode reaches a first voltage target value in the first pump cell control processing, the second pump cell such that a second voltage between the reference electrode and the second inner electrode reaches a second voltage target value in the second pump cell control processing, and the third pump cell such that a third voltage between the reference electrode and the third inner electrode reaches a third voltage target value in the third pump cell control processing.

### BRIEF DESCRIPTION OF THE DRAWINGS

FIG. 1 is a schematic cross-sectional view schematically showing an example of a configuration of a gas sensor 100.

FIG. 2 is a block diagram showing the electrical connections between a control device 95 and respective cells and other components.

FIG. 3 is a flowchart showing one example of the concentration derivation processing routine.

FIG. 4 is an explanatory diagram showing one example of a carbon dioxide concentration derivation map.

FIG. 5 is an explanatory diagram showing one example of a correction value derivation map.

FIG. 6 is a graph showing the relationship between a carbon dioxide concentration Ccd and the absolute value of the pump current Ip1.

FIG. 7 is an explanatory diagram showing one example of a water concentration derivation map.

FIG. 8 is a graph showing a relationship between a water concentration Cw and the absolute value of a pump current Ip1.

FIG. 9 is a schematic cross-sectional view of a sensor element 201 according to a modification.

### DETAILED DESCRIPTION OF THE INVENTION

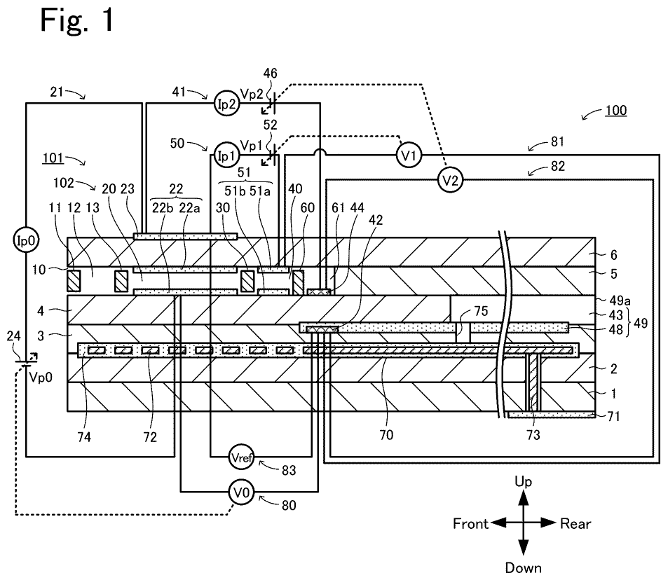

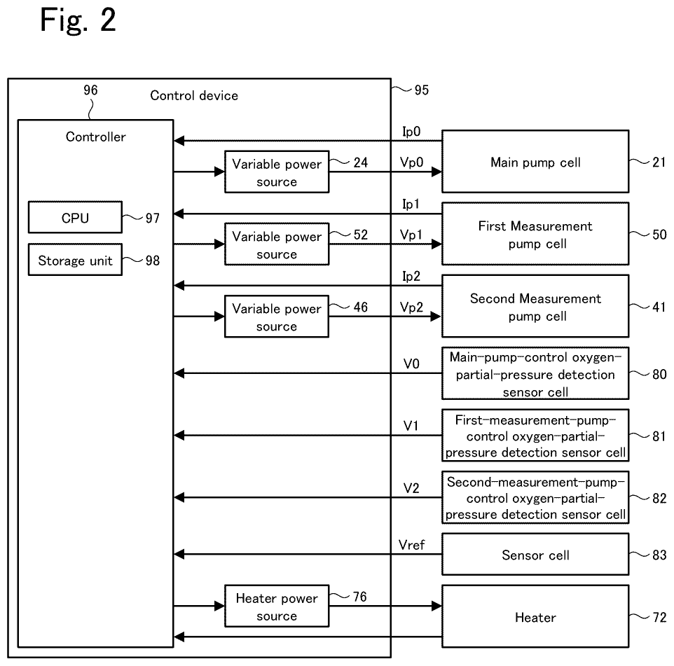

Next, embodiments of the present invention will be described with reference to the drawings. FIG. 1 is a schematic cross-sectional view schematically showing an example of a configuration of a gas sensor 100 according to an embodiment of the present invention. FIG. 2 is a block diagram showing the electrical connections between a control device 95, respective cells and a heater 72. The gas sensor 100 is installed in a pipe, such as an exhaust pipe of an internal combustion engine. The gas sensor 100 detects a concentration of a specific gas in a measurement gas, using exhaust gas from an internal combustion engine as the measurement gas. In the present embodiment, the gas sensor 100 is configured to detect the carbon dioxide concentration and the water concentration as the concentrations of the specific gases. The gas sensor 100 includes: a sensor element 101 with an elongated rectangular parallelepiped element body 102; cells 21, 41, 50, and 80 to 83 within the sensor element 101; a heater section 70 provided inside the sensor element 101; and a control device 95, which includes variable power sources 24, 46, and 52, and a heater power source 76, and controls the overall operation of the gas sensor 100. Note that the longitudinal direction (left-right direction in FIG. 1) of the sensor element 101 is defined as the front-rear direction, the thickness direction (up-down direction in FIG. 1) of the sensor element 101 as the up-down direction, and the width direction (perpendicular to both front-rear direction and up-down direction) of the sensor element 101 as the left-right direction.

The element body 102 is a laminated body in which six layers are stacked in the following order from the bottom in the drawing: a first substrate layer 1, a second substrate layer 2, a third substrate layer 3, a first solid electrolyte layer 4, a spacer layer 5, and a second solid electrolyte layer 6. Each of these layers is composed of an oxygen-ion-conductive solid electrolyte layer, such as zirconia (ZrO₂) or the like. The solid electrolytes forming these six layers are dense and hermetically sealed. The element body 102 is manufactured, for example, by performing predetermined processing and printing of circuit patterns on ceramic green sheets corresponding to the respective layers, laminating the sheets, and then firing the laminated sheets to integrate them into a unified structure.

On the front end side of the sensor element 101 (element body 102), between the lower surface of the second solid electrolyte layer 6 and the upper surface of the first solid electrolyte layer 4, the following components are formed adjacently and connected in sequence: a gas inlet 10; a first diffusion rate-limiting section 11; a buffer space 12; a second diffusion rate-limiting section 13; a first internal cavity 20; a third diffusion rate-limiting section 30; a second internal cavity 40; a fourth diffusion rate-limiting section 60; and a third internal cavity 61.

The gas inlet 10, buffer space 12, first internal cavity 20, second internal cavity 40, and third internal cavity 61 are internal spaces within the sensor element 101, formed by hollowing out portions of the spacer layer 5. These spaces are bounded at the top by the lower surface of the second solid electrolyte layer 6, at the bottom by the upper surface of the first solid electrolyte layer 4, and on the sides by the side surfaces of the spacer layer 5.

The first diffusion rate-limiting section 11, the second diffusion rate-limiting section 13, and the third diffusion rate-limiting section 30 are each provided as two horizontally elongated slits, with openings oriented along the longitudinal direction perpendicular to the plane of the drawing. The fourth diffusion rate-limiting section 60 is provided as a single horizontally elongated slit, with openings oriented along the longitudinal direction perpendicular to the plane of the drawing, formed as a gap with the lower surface of the second solid electrolyte layer 6. The area extending from the gas inlet 10 to the third internal cavity 61 is also referred to as the measurement gas flow path.

The sensor element 101 (element body 102) includes a reference gas introduction portion 49, which introduces a reference gas from outside of the sensor element 101 to a reference electrode 42 when measuring the concentrations of the specific gases. The reference gas introduction portion 49 comprises a reference gas introduction space 43 and a reference gas introduction layer 48. The reference gas introduction space 43 is an inward space formed from the rear end surface of the sensor element 101. The reference gas introduction space 43 is located between the upper surface of the third substrate layer 3 and the lower surface of the spacer layer 5, and is laterally defined by the side surfaces of the first solid electrolyte layer 4. The reference gas introduction space 43 opens to the rear end surface of the sensor element 101, with this opening serving as an inlet portion 49a of the reference gas introduction portion 49. The reference gas is introduced into the reference gas introduction space 43 through the inlet portion 49a. The reference gas introduction portion 49 introduces the reference gas, which has entered through the inlet portion 49a, to the reference electrode 42 while imparting a predetermined diffusion resistance. In the present embodiment, the reference gas is ambient air.

The reference gas introduction layer 48 is provided between the upper surface of the third substrate layer 3 and the lower surface of the first solid electrolyte layer 4. The reference gas introduction layer 48 is a porous body made of a ceramic material such as alumina or the like. A portion of the upper surface of the reference gas introduction layer 48 is exposed within the reference gas introduction space 43. The reference gas introduction layer 48 is formed so as to cover the reference electrode 42. The reference gas introduction layer 48 allows the reference gas to flow from the reference gas introduction space 43 to the reference electrode 42.

The reference electrode 42 is an electrode formed between the upper surface of the third substrate layer 3 and the first solid electrolyte layer 4, and as described above, the reference gas introduction layer 48, which is connected to the reference gas introduction space 43, is provided around the reference electrode 42. Furthermore, as will be explained later, the reference electrode 42 enables the measurement of the oxygen concentration (oxygen partial pressure) in the first internal cavity 20, the second internal cavity 40, and the third internal cavity 61. The reference electrode 42 is formed as a porous cermet electrode (for example, a cermet electrode of Pt and ZrO₂).

In the measurement gas flow path, the gas inlet 10 is a portion that is open to the external space, allowing the measurement gas to be drawn into the sensor element 101 from the external space. The first diffusion rate-limiting section 11 is a part that imparts a predetermined diffusion resistance to the measurement gas introduced through the gas inlet 10. The buffer space 12 is a space provided to guide the measurement gas introduced through the first diffusion rate-limiting section 11 to the second diffusion rate-limiting section 13. The second diffusion rate-limiting section 13 is a portion that imparts a predetermined diffusion resistance to the measurement gas introduced from the buffer space 12 into the first internal cavity 20. When the measurement gas is introduced from outside the sensor element 101 into the first internal cavity 20, the measurement gas that is abruptly drawn into the sensor element 101 through the gas inlet 10 due to pressure fluctuations in the external space (such as exhaust pulsations in the case where the measurement gas is automobile exhaust gas) is not directly introduced into the first internal cavity 20. Instead, after the pressure fluctuations of the measurement gas are attenuated through the first diffusion rate-limiting section 11, the buffer space 12, and the second diffusion rate-limiting section 13, the measurement gas is introduced into the first internal cavity 20. As a result, the pressure fluctuations of the measurement gas introduced into the first internal cavity 20 become almost negligible. The first internal cavity 20 is provided as a space for adjusting the oxygen partial pressure in the measurement gas introduced through the second diffusion rate-limiting section 13. This oxygen partial pressure is adjusted by the operation of a main pump cell 21.

The main pump cell 21 is an electrochemical pump cell, which is constituted by an inner pump electrode 22 with a ceiling electrode portion 22a provided on nearly the entire lower surface of the second solid electrolyte layer 6 facing the first internal cavity 20, an outer pump electrode 23, which is provided in a manner exposed to the outside of the sensor element 101 in a region of the upper surface of the second solid electrolyte layer 6 corresponding to the ceiling electrode portion 22a, and the second solid electrolyte layer 6, the spacer layer 5, and the first solid electrolyte layer 4, which form the current path between these electrodes.

The inner pump electrode 22 is formed so as to extend across the upper and lower solid electrolyte layers, (namely the second solid electrolyte layer 6 and the first solid electrolyte layer 4,) and the spacer layer 5 that provides sidewalls, which together define the first internal cavity 20. Specifically, the ceiling electrode portion 22a is formed on the lower surface of the second solid electrolyte layer 6, which constitutes the ceiling surface of the first internal cavity 20, and a bottom electrode portion 22b is formed on the upper surface of the first solid electrolyte layer 4, which constitutes the bottom surface of the first internal cavity 20. Further, in order to connect the ceiling electrode portion 22a and the bottom electrode portion 22b, side electrode portions (not shown) are formed on the side wall surfaces (inner surfaces) of the spacer layer 5, which constitute both sidewall portions of the first internal cavity 20. The inner pump electrode 22 is disposed in a tunnel-like structure at the region where the side electrode portion is provided.

The inner pump electrode 22 and the outer pump electrode 23 each are formed as a porous cermet electrode (for example, a cermet electrode of Pt and ZrO₂, having an Au content of 1%).

In the main pump cell 21, a desired voltage Vp0 is applied between the inner pump electrode 22 and the outer pump electrode 23, whereby a pump current Ip0 is caused to flow in a positive direction or a negative direction between the inner pump electrode 22 and the outer pump electrode 23. Thus, the oxygen in the first internal cavity 20 can be pumped out to the external space, or the oxygen in the external space can be pumped into the first internal cavity 20.

Further, in order to detect the oxygen concentration (oxygen partial pressure) in the atmosphere within the first internal cavity 20, an electrochemical sensor cell, that is, a main-pump-control oxygen-partial-pressure detection sensor cell 80, is constituted by the inner pump electrode 22, the second solid electrolyte layer 6, the spacer layer 5, the first solid electrolyte layer 4, the third substrate layer 3, and the reference electrode 42.

By measuring an electromotive force (voltage V0) in the main-pump-control oxygen-partial-pressure detection sensor cell 80, the oxygen concentration (oxygen partial pressure) in the first internal cavity 20 can be determined. Furthermore, by feedback-controlling the voltage Vp0 of the variable power source 24 such that the voltage V0 reaches a target value, the pump current Ip0 is controlled, thereby adjusting the oxygen concentration in the first internal cavity 20.

The third diffusion rate-limiting section 30 is a part that imparts a predetermined diffusion resistance to the measurement gas, whose oxygen concentration (oxygen partial pressure) is controlled by the operation of the main pump cell 21 in the first internal cavity 20, and guides the measurement gas into the second internal cavity 40.

The second internal cavity 40 is provided as a space in which the oxygen partial pressure is adjusted by the first measurement pump cell 50 for the measurement gas introduced through the third diffusion rate-limiting section 30, to carry out processing for measuring the water concentration in the measurement gas.

The first measurement pump cell 50 is an electrochemical pump cell, which is constituted by a first measurement electrode 51 with a ceiling electrode portion 51a provided on nearly the entire lower surface of the second solid electrolyte layer 6 facing the second internal cavity 40, the outer pump electrode 23 (not limited to the outer pump electrode 23, but may be any suitable electrode located outside the sensor element 101), the second solid electrolyte layer 6, the spacer layer 5, and the first solid electrolyte layer 4.

The first measurement electrode 51 is disposed within the second internal cavity 40 in a tunnel-like structure similar to that of the inner pump electrode 22 disposed in the first internal cavity 20 described above. Specifically, the ceiling electrode portion 51a is formed on the second solid electrolyte layer 6, which constitutes the ceiling surface of the second internal cavity 40, and a bottom electrode portion 51b is formed on the first solid electrolyte layer 4, which constitutes the bottom surface of the second internal cavity 40. Further, side electrode portions (not shown), which connect the ceiling electrode portion 51a and the bottom electrode portion 51b, are formed on the inner side surfaces of the spacer layer 5, which constitute both sidewall portions of the second internal cavity 40. Thus, the first measurement electrode 51 is formed in a tunnel-like structure. The first measurement electrode 51 is also formed as a porous cermet electrode using the same material as the inner pump electrode 22.

In the first measurement pump cell 50, a desired voltage Vp1 is applied between the first measurement electrode 51 and the outer pump electrode 23. Thus, the oxygen in the atmosphere within the second internal cavity 40 can be pumped out to the external space, or the oxygen can be pumped into the second internal cavity 40 from the external space.

Further, in order to control the oxygen partial pressure in the atmosphere within the second internal cavity 40, an electrochemical sensor cell, that is, an first-measurement-pump-control oxygen-partial-pressure detection sensor cell 81, is constituted by the first measurement electrode 51, the reference electrode 42, the second solid electrolyte layer 6, the spacer layer 5, the first solid electrolyte layer 4, and the third substrate layer 3.

The first measurement pump cell 50 performs pumping via the variable power source 52, which is voltage-controlled based on an electromotive force (voltage V1) detected by the first-measurement-pump-control oxygen-partial-pressure detection sensor cell 81. As a result, the oxygen partial pressure in the atmosphere of the second internal cavity 40 is adjusted by the pump current Ip1 flowing through the first measurement pump cell 50.

The fourth diffusion rate-limiting section 60 is a part that imparts a predetermined diffusion resistance to the measurement gas, whose oxygen concentration (oxygen partial pressure) is controlled by the operation of the first measurement pump cell 50 in the second internal cavity 40, and guides the measurement gas into the third internal cavity 61.

The third internal cavity 61 is provided as a space in which the oxygen partial pressure is adjusted by the second measurement pump cell 41 for the measurement gas introduced through the fourth diffusion rate-limiting section 60, to carry out processing for measuring the carbon dioxide concentration in the measurement gas.

The second measurement pump cell 41 is an electrochemical pump cell, which is constituted by a second measurement electrode 44 provided on the upper surface of the first solid electrolyte layer 4 facing the third internal cavity 61, the outer pump electrode 23, the second solid electrolyte layer 6, the spacer layer 5, and the first solid electrolyte layer 4. The second measurement electrode 44 is also formed as a porous cermet electrode using the same material as the inner pump electrode 22.

In the second measurement pump cell 41, a desired voltage Vp2 is applied between the second measurement electrode 44 and the outer pump electrode 23. Thus, the oxygen in the atmosphere within the third internal cavity 61 can be pumped out to the external space, or the oxygen can be pumped into the third internal cavity 61 from the external space.

Further, in order to detect the oxygen partial pressure around the second measurement electrode 44, an electrochemical sensor cell, that is, a second-measurement-pump-control oxygen-partial-pressure detection sensor cell 82, is formed by the first solid electrolyte layer 4, the third substrate layer 3, the second measurement electrode 44, and the reference electrode 42.

The variable power source 46 is controlled based on the electromotive force (voltage V2) detected by the second-measurement-pump-control oxygen-partial-pressure detection sensor cell 82, and the voltage Vp2 of the variable power source 46 is applied to the second measurement pump cell 41. As a result, the oxygen partial pressure in the atmosphere of the third internal cavity 61 is adjusted by the pump current Ip2 flowing through the second measurement pump cell 41.

Furthermore, an electrochemical sensor cell 83 is formed from the second solid electrolyte layer 6, the spacer layer 5, the first solid electrolyte layer 4, the third substrate layer 3, the outer pump electrode 23, and the reference electrode 42, and the oxygen partial pressure in the measurement gas outside the sensor can be detected based on the electromotive force (voltage Vref) obtained by this sensor cell 83.

The sensor element 101 includes a heater section 70 that performs temperature regulation by heating and maintaining the temperature of the sensor element 101, in order to enhance the oxygen-ion-conductivity of the solid electrolyte. The heater section 70 includes a heater connector electrode 71, a heater 72, a through hole 73, a heater insulating layer 74, and a pressure relief hole 75.

The heater connector electrode 71 is an electrode formed in such a manner as to be in contact with the lower surface of the first substrate layer 1. By connecting the heater connector electrode 71 to a heater power source 76 (see FIG. 2), power can be supplied from the heater power source 76 to the heater section 70.

The heater 72 is an electrical resistor formed in such a manner as to be sandwiched between the second substrate layer 2 and the third substrate layer 3 from above and below. The heater 72 is connected to the heater connector electrode 71 via the through hole 73, and generates heat when power is supplied from the heater power source 76 through the heater connector electrode 71, thereby heating and maintaining the temperature of the solid electrolyte forming the sensor element 101.

The heater 72 is also embedded across the entire region from the first internal cavity 20 to the third internal cavity 61, making it possible to adjust the temperature of the entire sensor element 101 to a level at which the solid electrolyte is activated.

The heater insulating layer 74 is an insulating layer formed of an insulator such as alumina and provided on the upper and lower surfaces of the heater 72. The heater insulating layer 74 is formed for the purpose of providing electrical insulation between the second substrate layer 2 and the heater 72, as well as between the third substrate layer 3 and the heater 72.

The pressure relief hole 75 is a portion that penetrates through the third substrate layer 3 and the reference gas introduction layer 48, and is formed so as to communicate with the reference gas introduction space 43. The pressure relief hole 75 is formed for the purpose of relieving an increase in internal pressure caused by a rise in temperature within the heater insulating layer 74.

As shown in FIG. 2, the control device 95 includes the variable power sources 24, 46, and 52 described above, the heater power source 76 also described above, and a control unit 96. The control unit 96 is a microprocessor including a CPU 97 and a storage unit 98. The storage unit 98 is a rewritable non-volatile memory, and is capable of storing, for example, various programs and various kinds of data. The control unit 96 inputs the voltage V0 from the main-pump-control oxygen-partial-pressure detection sensor cell 80, the voltage V1 from the first-measurement-pump-control oxygen-partial-pressure detection sensor cell 81, the voltage V2 from the second-measurement-pump-control oxygen-partial-pressure detection sensor cell 82, the voltage Vref from the sensor cell 83, the pump current Ip0 flowing through the main pump cell 21, the pump current Ip1 flowing through the first measurement pump cell 50, and the pump current Ip2 flowing through the second measurement pump cell 41. In addition, the control unit 96 controls the voltages Vp0, Vp1, and Vp2 output from the variable power sources 24, 46, and 52, respectively, by outputting control signals to the variable power sources 24, 46, and 52. Through this control, the main pump cell 21, the second measurement pump cell 41, and the first measurement pump cell 50 are controlled. The control unit 96 also controls the power supplied from the heater power source 76 to the heater 72 by outputting a control signal to the heater power source 76. The storage unit 98 also stores target values V0*, V1*, and V2*, which will be described later. The CPU 97 of the control unit 96 performs control of the respective cells 21, 41, and 50 with reference to the target values V0*, V1*, and V2*.

The control unit 96 performs a main pump control processing (an example of the first pump cell control processing), which controls the main pump cell 21 to pump out oxygen from around the inner pump electrode 22 to around the outer pump electrode 23. Specifically, the control unit 96 feedback-controls the voltage Vp0 of the variable power source 24 so that the voltage V0 reaches a target value V0*, thereby controlling the main pump cell 21. The target value V0* is set as a value such that the oxygen concentration in the first internal cavity 20 reaches a predetermined low concentration that is sufficiently low to substantially reduce all of the water and carbon dioxide in the measurement gas. By performing this main pump control processing, in the first internal cavity 20, water in the measurement gas is reduced to generate hydrogen and oxygen, and carbon dioxide in the measurement gas is reduced to generate carbon monoxide and oxygen. The generated oxygen is pumped out from around the inner pump electrode 22 to around the outer pump electrode 23 by the pump current Ip0 flowing through the main pump cell 21.

The control unit 96 performs a first measurement pump control processing (an example of the second pump cell control processing), which controls the first measurement pump cell 50 to pump into oxygen from around the outer pump electrode 23 to around the first measurement electrode 51. Specifically, the control unit 96 feedback-controls the voltage Vp1 of the variable power source 52 so that the voltage V1 reaches the target value V1*, thereby controlling the first measurement pump cell 50. The target value V1* is set as a value such that the oxygen concentration in the second internal cavity 40 reaches a predetermined concentration sufficient to substantially oxidize all of hydrogen in the second internal cavity 40. By performing this first measurement pump control processing, in the second internal cavity 40, hydrogen generated by the reduction of water in the first internal cavity 20 is oxidized to generate water again. At this time, the pump current Ip1 flowing through the first measurement pump cell 50 correlates with the amount of oxygen pumped into the second internal cavity 40 to oxidize the hydrogen in the second internal cavity 40, and hence correlates with the amount of water in the measurement gas in the first internal cavity 20, which is the source of the hydrogen in the second internal cavity 40. Therefore, the pump current Ip1 correlates with the water concentration in the measurement gas and the water concentration in the measurement gas can be detected based on the pump current Ip1. In the present embodiment, however, the control unit 96 corrects the pump current Ip1 based on the pump current Ip2 to derive a corrected pump current Ip1ad, and derives the water concentration based on the corrected pump current Ip1ad. Details of the correction will be described later.

The control unit 96 performs a second measurement pump control processing (an example of the third pump cell control processing), which controls the second measurement pump cell 41 to pump into oxygen from around the outer pump electrode 23 to around the second measurement electrode 44. Specifically, the control unit 96 feedback-controls the voltage Vp2 of the variable power source 46 so that the voltage V2 reaches the target value V2*, thereby controlling the second measurement pump cell 41. The target value V2* is set as a value such that the oxygen concentration in the third internal cavity 61 reaches a predetermined concentration sufficient to substantially oxidize all of carbon monoxide in the third internal cavity 61. By performing this second measurement pump control processing, in the third internal cavity 61, carbon monoxide generated by the reduction of carbon dioxide in the first internal cavity 20 is oxidized to generate carbon dioxide again. At this time, the pump current Ip2 flowing through the second measurement pump cell 41 correlates with the amount of oxygen pumped into the third internal cavity 61 to oxidize the carbon monoxide in the third internal cavity 61, and hence correlates with the amount of carbon dioxide in the measurement gas in the first internal cavity 20, which is the source of the carbon monoxide in the third internal cavity 61. Therefore, the pump current Ip2 correlates with the carbon dioxide concentration in the measurement gas and the carbon dioxide concentration in the measurement gas can be detected based on the pump current Ip2.

Both hydrogen and carbon monoxide generated in the first internal cavity 20 reach the second internal cavity 40. However, hydrogen has a faster gas diffusion rate than carbon monoxide and hydrogen binds with oxygen more readily than carbon monoxide does. Therefore, in the second internal cavity 40, hydrogen can be selectively oxidized, among hydrogen and carbon monoxide, by the first measurement pump control processing. Additionally, since hydrogen rarely reaches the third internal cavity 61 downstream of the second internal cavity 40, the second measurement pump control processing can oxidize carbon monoxide.

The control unit 96 performs heater control processing by outputting a control signal to the heater power source 76 such that the temperature of the heater 72 reaches to a target temperature (e.g. 800° C.). Here, the temperature of the heater 72 can be expressed as a linear function of the resistance value of the heater 72. In the heater control processing, the control unit 96 calculates the resistance value of the heater 72, which is a value can be regarded as the temperature of the heater 72 (a value that can be converted into temperature), and feedback-controls the heater power source 76 such that the calculated resistance value reaches to a target resistance value (a value corresponding to the target temperature). The control unit 96 can acquire the voltage of the heater 72 and the current flowing through the heater 72, then calculate the resistance of the heater 72 based on the acquired voltage and current. The control unit 96 may use a three-wire or four-wire method to calculate the resistance, for example. The heater power source 76 adjusts the power supplied to the heater 72 by changing the voltage applied to the heater 72 based on a control signal from the control unit 96 when energizing the heater 72.

In addition, the control device 95, which includes the variable power sources 24, 46, 52, and the heater power source 76 shown in FIG. 2, is actually connected to the electrodes inside the sensor element 101 through unillustrated lead wires formed within the sensor element 101 and unillustrated connector electrodes formed at the rear end of the sensor element 101 (only the heater connector electrode 71 shown in FIG. 1).

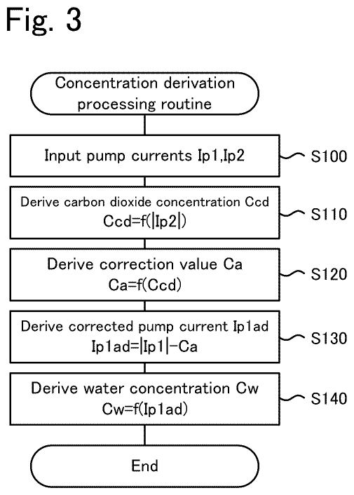

Next, an example of the processing by which the control unit 96 of the gas sensor 100 derives the concentrations of the specific gases (in this case, water concentration and carbon dioxide concentration) in the measurement gas will be described. The CPU 97 of the control unit 96 first performs the heater control processing described above to control the temperature of the heater 72 to reach a target temperature (e.g., 800° C.). Once the temperature of the heater 72 reaches (or approaches) the target temperature, the CPU 97 starts control of each of the pump cells 21, 41, 50 described above (i.e., the main pump control processing, first measurement pump control processing, and second measurement pump control processing) and acquires voltages V0, V1, V2, and Vref from the sensor cells 80 to 83 described above. Then, the CPU 97 derives the concentrations of the specific gases in the measurement gas based on the pump current Ip1 and the pump current Ip2. FIG. 3 is a flowchart showing an example of the concentration derivation processing routine performed by the CPU 97 of the control unit 96. The control unit 96 stores this routine in the storage unit 98, for example.

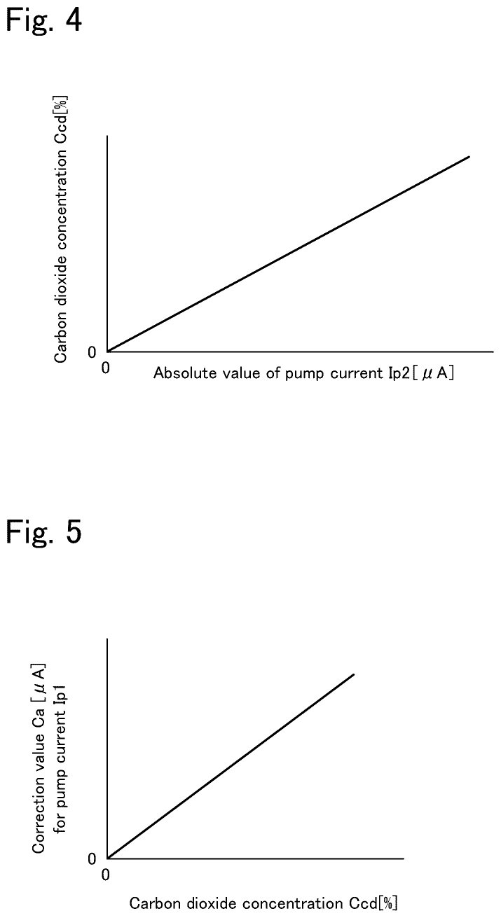

When the CPU 97 of the control unit 96 starts the concentration derivation processing routine, the CPU 97 first inputs the pump current Ip1 flowing through the first measurement pump cell 50 by the first measurement pump control processing, and the pump current Ip2 flowing through the second measurement pump cell 41 by the second measurement pump control processing (Step S100). Then, the CPU 97 derives the carbon dioxide concentration Ccd in the measurement gas based on the input value of pump current Ip2 (the detected value of pump current Ip2) (Step S110). The CPU 97 derives the carbon dioxide concentration Ccd, for example, using a carbon dioxide concentration derivation map. The carbon dioxide concentration derivation map is predetermined through experiments or analysis and so forth as a relationship between the absolute value of the pump current Ip2 and the carbon dioxide concentration Ccd and stored in the storage unit 98. FIG. 4 is an explanatory diagram showing one example of the carbon dioxide concentration derivation map. As described above, the pump current Ip2 flowing through the second measurement pump cell 41 by the second measurement pump control processing correlates with the carbon dioxide concentration Ccd in the measurement gas. For example, as shown in FIG. 4, there is a linear positive correlation between the absolute value of pump current Ip2 and carbon dioxide concentration Ccd. In Step S110, the CPU 97 applies the absolute value of pump current Ip2 input in Step S100 to the correspondence relationship in FIG. 4, and derives the carbon dioxide concentration Ccd corresponding to the absolute value of pump current Ip2. Thus, the carbon dioxide concentration in the measurement gas is measured.

Subsequently, the CPU 97 derives a correction value Ca based on the carbon dioxide concentration Ccd (Step S120), and derives a corrected pump current Ip1ad by correcting the value of the pump current Ip1 (the detected value of the pump current Ip1) input in Step S100 using this correction value Ca (Step S130). In the present embodiment, the correction value Ca is a subtraction value to the absolute value of the pump current Ip1, and the CPU 97 derives the corrected pump current Ip1ad by subtracting the correction value Ca from the absolute value of the pump current Ip1. The derivation of the correction value Ca in Step S120 is performed, for example, using a correction value derivation map. The correction value derivation map is predetermined through experiments or analysis and so forth as a correspondence relationship between the carbon dioxide concentration Ccd and the correction value Ca and stored in the storage unit 98. FIG. 5 is an explanatory diagram showing one example of the correction value derivation map. As shown in FIG. 5, there is a linear positive correlation between the carbon dioxide concentration Ccd and the correction value Ca, and the higher the carbon dioxide concentration Ccd, the greater the correction value Ca is set to be. In addition, the correction value Ca corresponding to the case where carbon dioxide concentration Ccd of 0% is set to a value of 0, and in the case of the carbon dioxide concentration Ccd of 0%, the absolute value of the pump current Ip1 is equal to the corrected pump current Ip1ad. In Step S120, the CPU 97 applies the carbon dioxide concentration Ccd derived in Step S110 to the correspondence relationship shown in FIG. 5 and derives the correction value Ca corresponding to the carbon dioxide concentration Ccd. Therefore, in Step S130, the correction is performed such that the higher the carbon dioxide concentration Ccd, the more the absolute value of the pump current Ip1 tends to decrease, and the corrected pump current Ip1ad is derived.

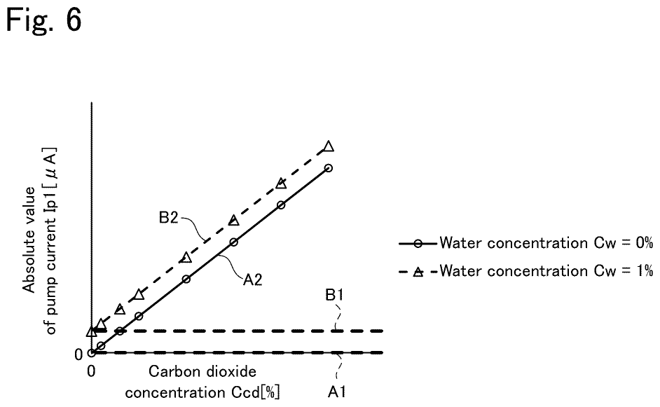

The reason for deriving the corrected pump current Ip1ad by correcting the absolute value of the pump current Ip1 using the correction value Ca derived from the correspondence relationship shown in FIG. 5 will be described. FIG. 6 is a graph showing the relationship between the carbon dioxide concentration Ccd [%] and the absolute value of the pump current Ip1 [μA] when the carbon dioxide concentration Ccd is varied with the water concentration Cw in the measurement gas fixed at a constant value. In FIG. 6, a straight line A1 represents an ideal graph in the case where the water concentration Cw is fixed at 0%, and a straight line A2 represents a graph based on actual measurement values. Similarly, a straight line B1 represents an ideal graph in the case where the water concentration Cw is fixed at 1%, and a straight line B2 represents a graph based on actual measurement values. The straight lines A2 and B2 were derived as approximate lines based on eight actual measurement points (see circles and triangles in the figure) shown in FIG. 6. As described above, since hydrogen has a higher gas diffusion rate than carbon monoxide and more readily combines with oxygen, in the second internal cavity 40, hydrogen can be selectively oxidized out of hydrogen and carbon monoxide by the first measurement pump control processing. Therefore, the pump current Ip1 correlates with the water concentration Cw, and ideally, the pump current Ip1 should not change depending on the carbon dioxide concentration Ccd. Accordingly, ideally, as shown by the straight lines A1 and B1, if the water concentration Cw is constant, the absolute value of the pump current Ip1 should remain constant even when the carbon dioxide concentration Ccd changes. However, in practice, both the straight lines A2 and B2 show that even when the water concentration Cw is constant the absolute value of the pump current Ip1 varies depending on the carbon dioxide concentration Ccd. Specifically, it has been confirmed that the higher the carbon dioxide concentration Ccd, the greater the absolute value of the pump current Ip1 tends to become. In other words, when the carbon dioxide concentration Ccd is 0%, the ideal value and the actual absolute value of the pump current are equal, but it has been confirmed that the deviation between the ideal values (the straight lines A1 and B1) and the actual absolute value of the pump current Ip1 tends to increase as the carbon dioxide concentration Ccd increases. The reason for this is considered to be that the higher the carbon dioxide concentration Ccd in the measurement gas, the more carbon monoxide is generated by the reduction of carbon dioxide in the first internal cavity 20, and part of the carbon monoxide is oxidized not in the third internal cavity 61 but in the second internal cavity 40 upstream of the third internal cavity 61. As a result, in the second measurement pump control processing, oxygen is pumped into not only for oxidizing hydrogen but also for oxidizing carbon monoxide, and thus the absolute value of the pump current Ip1 is considered to increase. In this manner, since the absolute value of the pump current Ip1 increases depending on the carbon dioxide concentration Ccd, there is a concern that simply deriving the water concentration Cw from the absolute value of the pump current Ip1 would result in a decrease in the measurement accuracy of the water concentration Cw. For this reason, in the present embodiment, as described above, the absolute value of the pump current Ip1 is corrected using the correction value Ca derived based on the carbon dioxide concentration Ccd, and the corrected pump current Ip1ad is derived.

Further, as can be seen from FIG. 6, the straight lines A2 and B2 had almost the same slope. That is, in both cases where the water concentration Cw is 0% and 1%, the amount of increase in the absolute value of the pump current Ip1 from the ideal value Ip1, in accordance with the value of carbon dioxide concentration Ccd, was nearly the same. From this result, it can be understood that, if the correction value Ca is defined as a subtraction value corresponding to the carbon dioxide concentration Ccd so as to return (correct) the absolute value of the pump current Ip1, which has increased due to the influence of the carbon dioxide concentration Ccd, to the ideal value, the absolute value of the pump current Ip1 can be corrected with relatively high accuracy using the same correction value Ca, regardless of the magnitude of the water concentration Cw (that is, regardless of the magnitude of the absolute value of the pump current Ip1). Therefore, in the present embodiment, a straight line having the same slope as that of the straight lines A2 and B2 and passing through the origin is used as the correspondence relationship between the carbon dioxide concentration Ccd and the correction value Ca, that is, as the correspondence relationship shown in FIG. 5. Note that, since the straight line A2 in FIG. 6 itself is a straight line passing through the origin, the straight line A2 can be directly used as the correspondence relationship shown in FIG. 5 described above. Using the correction value derivation map thus obtained, the correction value Ca is derived in Step S120, and the correction in Step S130 is performed using this correction value Ca. As a result, the influence of the carbon dioxide concentration Ccd on the absolute value of the pump current Ip1 can be reduced, and the corrected pump current Ip1ad can be derived as the absolute value of the pump current Ip1 that more accurately corresponds to the actual water concentration Cw in the measurement gas. Furthermore, by using the correction value Ca as a subtraction value for the absolute value of the pump current Ip1, it is possible to derive the corrected pump current Ip1ad by correcting the absolute value of the pump current Ip1 with relatively high accuracy using the same correction value Ca, regardless of the magnitude of the water concentration Cw (regardless of the magnitude of the absolute value of the pump current Ip1).

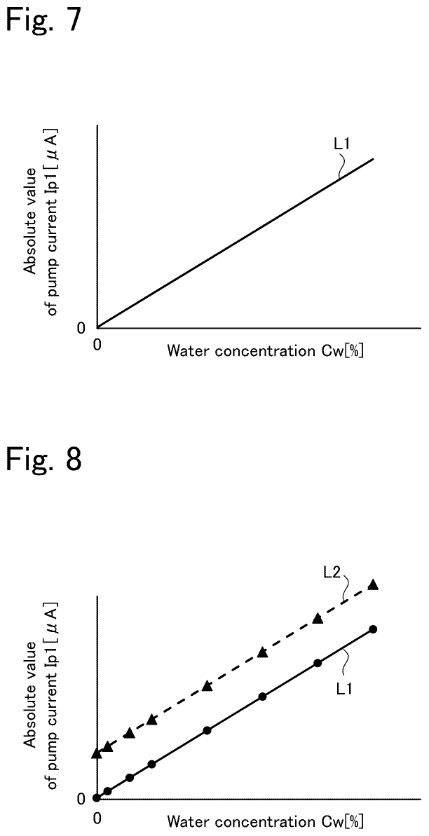

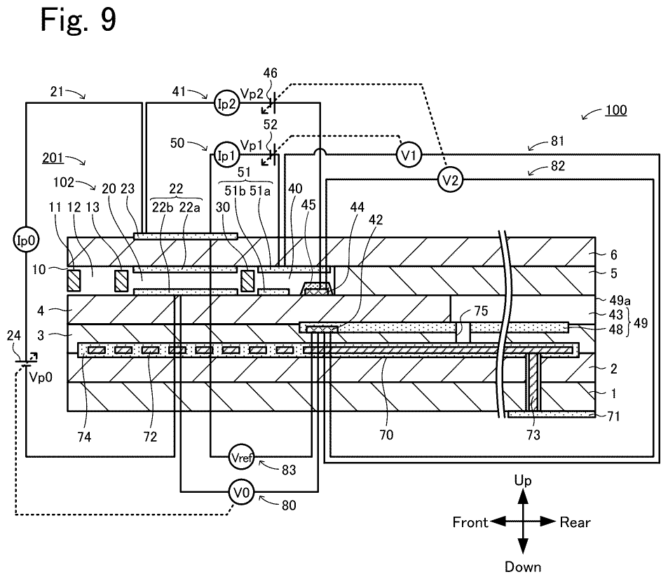

When the corrected pump current Ip1ad is derived in Step S130, the CPU 97 derives the water concentration Cw in the measurement gas based on the corrected pump current Ip1ad (Step S140), and then ends the routine. In Step S140, the CPU 97 derives the water concentration Cw using, for example, a water concentration derivation map. The water concentration derivation map is predetermined through experiments or analysis and so forth as a relationship between the absolute value of the pump current Ip1 and the water concentration Cw and stored in the storage unit 98. FIG. 7 is an explanatory diagram showing one example of the water concentration derivation map. As described above, the pump current Ip1 flowing through the first measurement pump cell 50 by the first measurement pump control processing correlates with the water concentration Cw in the measurement gas, and, for example, as shown by the straight line L1 in FIG. 7, there is a linear positive correlation between the absolute value of the pump current Ip1 and the water concentration Cw. However, this straight line L1 represents the correspondence relationship between the absolute value of the pump current Ip1 and the water concentration Cw in the case where the carbon dioxide concentration Ccd in the measurement gas is 0%, that is, a graph obtained by investigating the ideal correspondence relationship between the absolute value of the pump current Ip1 and the water concentration Cw. In Step S140, the CPU 97 applies the corrected pump current Ip1ad derived in Step S130 to the correspondence relationship shown by the straight line L1 in FIG. 7, in other words, uses the value of the corrected pump current Ip1ad as the absolute value of the pump current Ip1, and derives the water concentration Cw corresponding to this value. In this manner, by deriving the water concentration Cw using the corrected pump current Ip1ad, instead of the absolute value of the pump current Ip1 input in Step S100, it is possible to suppress a decrease in the measurement accuracy of the water concentration Cw caused by the carbon dioxide in the measurement gas.

A description will be given of the difference in measurement accuracy of the water concentration Cw between a case where the absolute value of the pump current Ip1 input in Step S100 is used without correction, and a case where the corrected pump current Ip1ad is used. FIG. 8 is a graph showing the relationship between the water concentration Cw [%] and the absolute value of the pump current Ip1 [μA]. In FIG. 8, the straight line L1 from FIG. 7 is also shown. The eight triangular data points in FIG. 8 plot the relationship between the water concentration Cw (not the value derived by the gas sensor 100 but the true value of the water concentration Cw in the model gas) and the absolute value of the pump current Ip1 input in Step S100, under conditions where the carbon dioxide concentration Ccd has a nonzero value (e.g., 1%), as investigated using a model gas. In this way, when the measurement gas contains carbon dioxide, the correspondence relationship between the uncorrected absolute value of the pump current Ip1 and the true water concentration Cw becomes like a straight line L2 that is deviated from the straight line L1. This deviation is the same as the deviation shown in FIG. 6 between the straight lines A1, B1 and the straight lines A2, B2 (i.e., increase in the absolute value of the pump current Ip1). Due to this deviation, if the absolute value of the pump current Ip1 input in Step S100 is applied to the correspondence relationship defined by straight line L1 to derive the water concentration Cw, the derived water concentration Cw tends to be larger than the true water concentration Cw, resulting in decreased measurement accuracy. In contrast, data corrected by subtracting the above-mentioned correction value Ca from the absolute values of the pump current Ip1 of the eight data points shown by triangles in FIG. 8 is shown by black circles in FIG. 8. As shown, the black circles lie almost on the same straight line as the straight line L1. From this result, it has been confirmed that the correspondence relationship between the corrected absolute value of the pump current Ip1, that is, the corrected pump current Ip1ad, and the true water concentration Cw is nearly the same as the straight line L1. Therefore, by applying the corrected pump current Ip1ad to the correspondence relationship defined by the straight line L1 to derive the water concentration Cw, the derived water concentration Cw becomes almost equal to the true water concentration Cw, and it has been confirmed that the water concentration Cw can be measured with high accuracy.

Here, if the pump current Ip2 varies not only with the carbon dioxide concentration Ccd but also with the water concentration Cw, there is a concern that the correlation between the absolute value of the pump current Ip2 and the carbon dioxide concentration Ccd, which serves as the basis for deriving the correction value Ca, may be disrupted. For example, when the water concentration Cw is higher than the carbon dioxide concentration Ccd, the reduction of carbon dioxide in the first internal cavity 20 may become insufficient, and even if the carbon dioxide concentration Ccd is the same, the pump current Ip2 may decrease depending on the water concentration Cw. However, the higher the carbon dioxide concentration Ccd is relative to the water concentration Cw, the lower such concern becomes. In addition, as described above, the decrease in the measurement accuracy of the water concentration Cw caused by carbon dioxide in the measurement gas tends to become more pronounced as the carbon dioxide concentration Ccd in the measurement gas becomes higher. Therefore, the gas sensor 100 of the present embodiment is particularly suitable for detecting the water concentration Cw in cases where the carbon dioxide concentration Ccd in the measurement gas is higher than the water concentration Cw, especially in cases where the carbon dioxide concentration Ccd is sufficiently higher than the water concentration Cw such that the reduction of carbon dioxide in the first internal cavity 20 does not become insufficient.

As described above, the higher the carbon dioxide concentration Ccd in the measurement gas, the greater the amount of carbon monoxide generated by the reduction of carbon dioxide in the first internal cavity 20, and a part of the carbon monoxide is oxidized not in the third internal cavity 61 but in the second internal cavity 40 upstream therefrom. Therefore, compared to a case where all of the carbon monoxide generated by the reduction of carbon dioxide in the first internal cavity 20 is oxidized in the third internal cavity 61, the pump current Ip2 decreases in accordance with the amount of carbon monoxide oxidized in the second internal cavity 40. However, if such a decrease in the pump current Ip2 is reflected in the derivation of the carbon dioxide concentration Ccd based on the pump current Ip2 in Step S110, the carbon dioxide concentration Ccd can be accurately derived based on the pump current Ip2. For example, when a carbon dioxide concentration map such as shown in FIG. 4 is determined through experiments using a model gas, the correspondence relationship between the carbon dioxide concentration Ccd in the model gas and the absolute value of the pump current Ip2 obtained through the experiments necessarily reflects the decrease in the pump current Ip2 caused by the amount of carbon monoxide oxidized in the second internal cavity 40. Therefore, if the carbon dioxide concentration derivation map is determined based on the correspondence relationship obtained through the experiments, the carbon dioxide concentration Ccd can be accurately derived based on the pump current Ip2.

Here, the correspondence relationship between the elements according to the present embodiment and the elements according to the present invention will be clarified. The sensor element 101 according to the present embodiment corresponds to the sensor element according to the present invention. The element body 102 corresponds to the element body. The first internal cavity 20 corresponds to the first chamber. The inner pump electrode 22 corresponds to the first inner electrode. The main pump cell 21 corresponds to the first pump cell. The second internal cavity 40 corresponds to the second chamber. The first measurement electrode 51 corresponds to the second inner electrode. The first measurement pump cell 50 corresponds to the second pump cell. The third internal cavity 61 corresponds to the third chamber. The second measurement electrode 44 corresponds to the third inner electrode. The second measurement pump cell 41 corresponds to the third pump cell. The outer pump electrode 23 corresponds to the first outer electrode, the second outer electrode, and the third outer electrode. The control device 95 corresponds to the control device. The main pump control processing corresponds to the first pump cell control processing. The first measurement pump control processing corresponds to the second pump cell control processing. The second measurement pump control processing corresponds to the third pump cell control processing. The pump current Ip2 corresponds to the third pump current. The pump current Ip1 corresponds to the second pump current. The absolute value of the pump current Ip1 corresponds to the absolute value of the second pump current. The absolute value of the pump current Ip2 corresponds to the absolute value of the third pump current. The corrected pump current Ip1ad corresponds to the corrected second pump current. The correction value Ca corresponds to the correction value. The reference electrode 42 corresponds to the reference electrode. The voltage V0 corresponds to the first voltage. The target value V0* corresponds to the first voltage target value. The voltage V1 corresponds to the second voltage. The target value V1* corresponds to the second voltage target value. The voltage V2 corresponds to the third voltage. The target value V2* corresponds to the third voltage target value.

According to the gas sensor 100 of the present embodiment described in detail above, the control device 95 derives the water concentration Cw in the measurement gas based on the pump current Ip1, while taking into account the pump current Ip2. More specifically, the control device 95 derives the corrected pump current Ip1ad by correcting the pump current Ip1 taking into account the pump current Ip2, and then derives the water concentration Cw based on the derived corrected pump current Ip1ad. Still more specifically, the control device 95 derives the carbon dioxide concentration Ccd based on the absolute value of the pump current Ip2, derives the correction value Ca based on the derived carbon dioxide concentration Ccd, and corrects the pump current Ip1 using the derived correction value Ca, thereby deriving the corrected pump current Ip1ad. Then, the control device 95 derives the water concentration Cw based on the corrected pump current Ip1ad. As a result, the decrease in the measurement accuracy of the water concentration Cw caused by the carbon dioxide in the measurement gas can be suppressed.

In addition, the control device 95 derives the corrected pump current Ip1ad by applying a correction in such a manner that the greater the absolute value of the pump current Ip2, the more the absolute value of the pump current Ip1 tends to decrease. More specifically, the control device 95 derives a larger carbon dioxide concentration Ccd as the absolute value of the pump current Ip2 increases, and applies a correction such that the greater the derived carbon dioxide concentration Ccd, the more the absolute value of the pump current Ip1 tends to decrease, thereby deriving the corrected pump current Ip1ad. By performing a correction according to such a tendency, it becomes possible to more reliably suppress a decrease in the measurement accuracy of the water concentration Cw caused by the carbon dioxide in the measurement gas.

Furthermore, the correction value Ca is a subtraction value for the absolute value of the pump current Ip1, and the control device 95 corrects the pump current Ip1 by subtracting the correction value Ca from the absolute value of the pump current Ip1. The control device 95 corrects the pump current Ip1 using the same correction value Ca derived based on the absolute value of the pump current Ip2, regardless of the magnitude of the absolute value of the pump current Ip1. In this way, when the correction value Ca is a subtraction value, relatively accurate correction of the pump current Ip1 can be achieved even using the same correction value Ca based on the absolute value of the pump current Ip2, regardless of the magnitude of the absolute value of the pump current Ip1. Therefore, the correction can be performed more easily compared to the case where the correction value Ca is derived based on not only the absolute value of the pump current Ip2 but also the absolute value of the pump current Ip1.

Furthermore, since the control device 95 also derives the carbon dioxide concentration Ccd in the measurement gas based on the pump current Ip2, the gas sensor 100 can derive not only the water concentration Cw but also the carbon dioxide concentration Ccd.

It should be noted that the present invention is not limited to the present embodiment described above in any way, and it goes without saying that the present invention can be implemented in various modes as long as they fall within the technical scope of the present invention.

For example, in the above-described embodiment, the control device 95 derives the carbon dioxide concentration Ccd based on the pump current Ip2, but the derivation of the carbon dioxide concentration Ccd may be omitted. As described above, since the absolute value of the pump current Ip2 correlates with the carbon dioxide concentration Ccd, it is possible to derive the water concentration Cw by performing correction on the pump current Ip1 using the absolute value of the pump current Ip2 instead of the carbon dioxide concentration Ccd, in the same manner as in the above-described embodiment. For example, the control device 95 may omit Step S110 in FIG. 3 and, instead of Step S120, derive the correction value Ca based on the absolute value of the pump current Ip2. In this case, instead of the correction value derivation map shown in FIG. 5, a correction value derivation map that defines, in advance by experiments or analysis and so forth, the correspondence relationship between the absolute value of the pump current Ip2 and the correction value Ca may be used. This correction value derivation map may also be defined, for example, based on FIGS. 4 and 5.

In the above-described embodiment, the correction value Ca is defined as a subtraction value from the absolute value of the pump current Ip1, but the present invention is not limited thereto. For example, the correction value Ca may be a multiplication coefficient (a value between 0 and 1) for the absolute value of the pump current Ip1. In this case, as can be seen from the comparison between the straight lines A2 and B2 in FIG. 6, the correction value to be multiplied in order to restore (correct) the absolute value of the pump current Ip1 to the ideal value varies not only with the absolute value of the pump current Ip2 (or the carbon dioxide concentration Ccd based thereon), but also with the water concentration Cw, that is, the value of the absolute value of the pump current Ip1. Therefore, in a case where the correction value Ca is a multiplication coefficient applied to the absolute value of the pump current Ip1, it is preferable to use a correction value derivation map, which defines, in advance by experiments or analysis and so forth, the correspondence relationship among the absolute value of the pump current Ip2 (or the carbon dioxide concentration Ccd based thereon), the absolute value of the pump current Ip1, and the correction value Ca, instead of the correction value derivation map shown in FIG. 5. Even in a case where the correction value Ca is a subtraction value from the absolute value of the pump current Ip1, as in the above-described embodiment, it is also acceptable to use a correction value derivation map that defines the correspondence relationship among the absolute value of the pump current Ip2 (or the carbon dioxide concentration Ccd based thereon), the absolute value of the pump current Ip1, and the correction value Ca.

In the above-described embodiment, as shown in FIG. 5, the correction value derivation map is a map that represents a linear correspondence relationship between the carbon dioxide concentration Ccd and the correction value Ca, however, the present invention is not limited thereto. For example, the correction value derivation map may be configured to perform correction such that the greater the absolute value of the pump current Ip2 (or the carbon dioxide concentration Ccd based thereon), the more the absolute value of the pump current Ip1 tends to decrease, and the correspondence relationship between the carbon dioxide concentration Ccd and the correction value Ca may be a curved correspondence relationship or a step-function correspondence relationship. In addition, instead of the map as shown in FIG. 5, a relational expression (mathematical expression) representing the correspondence relationship between the absolute value of the pump current Ip2 (or the carbon dioxide concentration Ccd based thereon) and the correction value Ca may be used. Similarly, the carbon dioxide concentration derivation map in FIG. 4 and the water concentration derivation map in FIG. 7 may also represent a curved or step-function correspondence relationship, and a relational expression (or mathematical expression) may be used instead of the map. Moreover, the correction value derivation map is not limited to one that applies the correction such that the greater the absolute value of the pump current Ip2 (or the carbon dioxide concentration Ccd based thereon), the more the absolute value of the pump current Ip1 tends to decrease. For example, depending on conditions, there may be cases in which the absolute value of the pump current Ip1 decreases as the absolute value of the pump current Ip2 (or the carbon dioxide concentration Ccd based thereon) increases. In such cases where the sensor element 101 is used under such conditions, it is also acceptable to use a correction value derivation map that performs correction such that the greater the absolute value of the pump current Ip2 (or the carbon dioxide concentration Ccd based thereon), the more the absolute value of the pump current Ip1 tends to increase.

In the above-described embodiment, the control device 95 derives a corrected pump current Ip1ad by performing a correction on the pump current Ip1, taking into account the carbon dioxide concentration Ccd derived from the pump current Ip2, and derives the water concentration Cw based on the corrected pump current Ip1ad. However, the present invention is not limited thereto. For example, the control device 95 may derive a provisional water concentration Cwt based on the pump current Ip1, and then derive the water concentration Cw by correcting the provisional water concentration Cwt, taking into account the pump current Ip2. Even in this case, since the water concentration Cw is derived based on the pump current Ip1 by taking into account the pump current Ip2, as in the above-described embodiment, it is possible to suppress a decrease in the measurement accuracy of the water concentration Cw caused by carbon dioxide in the measurement gas. For example, when deriving the provisional water concentration Cwt based on the pump current Ip1, the control device 95 may derive the provisional water concentration Cwt as the water concentration obtained by applying the absolute value of the pump current Ip1 to the water concentration derivation map shown in FIG. 7. Further, when deriving the water concentration Cw based on the provisional water concentration Cwt by taking into account the absolute value of the pump current Ip2 (or the carbon dioxide concentration Ccd based thereon), the water concentration Cw may be derived by applying a correction in such a manner that the greater the absolute value of the pump current Ip2 (or the carbon dioxide concentration Ccd based thereon), the more the provisional water concentration Cwt tends to decrease. In this case, for example, a correction value Cb may be derived based on the absolute value of the pump current Ip2 (or the carbon dioxide concentration Ccd based thereon), and the provisional water concentration Cwt may be corrected using the correction value Cb. The correction value Cb is used to correct the deviation between the provisional water concentration Cwt and the water concentration Cw caused by the carbon dioxide concentration Ccd, and may be derived using a map similar to the correction value derivation map for correction value Ca shown in FIG. 5 (for example, a map in which the unit of the correction value Ca in FIG. 5 is converted from [μA] to [%]). Alternatively, a map that is different from FIG. 5 and represent the correspondence relationship between the absolute value of the pump current Ip2 (or the carbon dioxide concentration Ccd based thereon) and the correction value Cb may be defined in advance, by experiments or analysis and so forth. The correction value Cb may be a subtraction value applied to the provisional water concentration Cwt. In this case, the corrected provisional water concentration Cwt, that is, the water concentration Cw, may be derived by subtracting the correction value Cb from the provisional water concentration Cwt. When the correction value Cb is used as the subtraction value, the provisional water concentration Cwt may be corrected using the same correction value Cb derived based on the absolute value of the pump current Ip2 (or the carbon dioxide concentration Ccd based thereon), regardless of the magnitude of the provisional water concentration Cwt. Alternatively, the correction value Cb may be derived using a correction value derivation map that defines, in advance by experiments or analysis and so forth, the correspondence relationship among the absolute value of the pump current Ip2 (or the carbon dioxide concentration Ccd based thereon), the provisional water concentration Cwt, and the correction value Cb. Furthermore, various modifications similar to those described for the correction value Ca may also be applied to the correction value Cb.

In the above-described embodiment, the carbon dioxide concentration Ccd is derived based on the pump current Ip2, the correction value Ca is derived based on the derived carbon dioxide concentration Ccd, the absolute value of the pump current Ip1 is corrected using the derived correction value Ca, thereby the corrected pump current Ip1ad is derived, and the water concentration Cw is derived based on the corrected pump current Ip1ad. In the modification, the provisional water concentration Cwt is derived based on the pump current Ip1, and then the water concentration Cw is derived by correcting the provisional water concentration Cwt based on the pump current Ip2. However, the present invention is not limited thereto, it is sufficient as long as the water concentration Cw is derived based on the pump current Ip1, while taking into account the pump current Ip2. For example, the water concentration Cw may be derived by applying both the pump current Ip1 and the pump current Ip2 to a predetermined correspondence relationship among the pump current Ip1, the pump current Ip2, and the water concentration Cw. This correspondence relationship can be defined, for example, based on FIGS. 4 to 7. If there is the relationship such that the greater the absolute value of the pump current Ip2 (or the carbon dioxide concentration Ccd based thereon), the smaller the absolute value of the pump current Ip1 becomes as described above, a correspondence relationship different from those in FIGS. 4 to 8 may be used.

In the above-described embodiment, the outer pump electrode 23 plays a role as the first outer electrode to be paired with the inner pump electrode 22 of the main pump cell 21, a role as the second outer electrode to be paired with the first measurement electrode 51 of the first measurement pump cell 50, and a role as the third outer electrode to be paired with the second measurement electrode 44 of the second measurement pump cell 41. In other words, the first to third outer electrodes are configured as a common outer pump electrode 23. However, the present invention is not limited thereto. For example, two of the first to third outer electrodes may be configured as a common electrode, and the remaining one may be provided outside the element body 102 as an electrode independent of the outer pump electrode 23 so as to be in contact with the measurement gas. Alternatively, the first to third outer electrodes may each be provided as independent electrodes outside the element body 102 so as to be in contact with the measurement gas.

In the above-described embodiment, the sensor element 101 of the gas sensor 100 includes the first internal cavity 20, the second internal cavity 40, and the third internal cavity 61. However, the present invention is not limited thereto. For example, as shown in FIG. 10, a sensor element 201 according to a modification may not include the third internal cavity 61. In the sensor element 201 according to a modification shown in FIG. 10, between the lower surface of the second solid electrolyte layer 6 and the upper surface of the first solid electrolyte layer 4, the following components are adjacently formed and in communication with each other, in the order listed: a gas inlet 10; a first diffusion rate-limiting section 11; a buffer space 12; a second diffusion rate-limiting section 13; a first internal cavity 20; a third diffusion rate-limiting section 30; and a second internal cavity 40. In addition, the second measurement electrode 44 is disposed on the upper surface of the first solid electrolyte layer 4 within the second internal cavity 40. The second measurement electrode 44 is covered with a fourth diffusion rate-limiting section 45, which is a film made of a porous ceramic material such as alumina (Al₂O₃). The fourth diffusion rate-limiting section 45, similarly to the fourth diffusion rate-controlling section 60 of the above-described embodiment, serves to impart a predetermined diffusion resistance to the measurement gas in the second internal cavity 40 and guide the measurement gas to the second measurement electrode 44. Furthermore, the fourth diffusion rate-limiting section 45 also functions as a protective film for the measurement electrode 44. The ceiling electrode portion 51a of the first measurement electrode 51 extends directly above the second measurement electrode 44. Even with such a configuration of the sensor element 201, the carbon dioxide concentration Ccd can be detected based on the pump current Ip2 flowing through the second measurement pump cell 41, similarly to the embodiment described above. In the sensor element 201 of FIG. 10, the region around the second measurement electrode 44 functions as the third chamber. That is, the area around the second measurement electrode 44 serves the same role as the third internal cavity 61.

In the above-described embodiment, the element body 102 of the sensor element 101 is formed as the laminated body including multiple solid electrolyte layers (layers 1 to 6). However, the present invention is not limited thereto. The element body of the sensor element 101 only needs to include at least one oxygen ion-conductive solid electrolyte layer, and have a measurement gas flow path therein. For example, in FIG. 1, layers 1 to 5, except for the second solid electrolyte layer 6, may be structural layers made of materials other than solid electrolyte (e.g., layers made of alumina). In such a case, each electrode included in the sensor element 101 may be disposed on the second solid electrolyte layer 6. For instance, the second measurement electrode 44 shown in FIG. 1 may be disposed on a lower surface of the second solid electrolyte layer 6. Furthermore, the reference gas introduction space 43, which is formed in the first solid electrolyte layer 4, may instead be formed in the spacer layer 5. Likewise, the reference gas introduction layer 48, located between the first solid electrolyte layer 4 and the third substrate layer 3, may instead be provided between the second solid electrolyte layer 6 and the spacer layer 5. In addition, the reference electrode 42 may be provided on the lower surface of the second solid electrolyte layer 6 at a position downstream of the third internal cavity 61.

In the above-described embodiment, the first diffusion rate-limiting section 11, the second diffusion rate-limiting section 13, and the third diffusion rate-limiting section 30 are each provided as two horizontally elongated slits, but the present invention is not limited thereto. For example, one or more of the first diffusion rate-limiting section 11, the second diffusion rate-limiting section 13, and the third diffusion rate-limiting section 30 may be configured as a single horizontal elongated slit.

What is claimed is:

1. A gas sensor comprising a sensor element and a control device, and configured to measure a water concentration in a measurement gas,
wherein:
the sensor element includes:
an element body having an oxygen-ion-conductive solid electrolyte layer and a measurement gas flow path formed therein for introducing and allowing flow of the measurement gas;
a first pump cell including a first inner electrode disposed in a first chamber of the measurement gas flow path, and a first outer electrode disposed on the outside of the element body;
a second pump cell including a second inner electrode disposed in a second chamber located downstream of the first chamber of the measurement gas flow path, and a second outer electrode disposed on the outside of the element body;
a third pump cell including a third inner electrode disposed in a third chamber located downstream of the second chamber of the measurement gas flow path, and a third outer electrode disposed on the outside of the element body;
the control device performs:
a first pump cell control processing in which oxygen is pumped out from around the first inner electrode to around the first outer electrode by controlling the first pump cell, thereby reducing water and carbon dioxide in the measurement gas in the first chamber;
a second pump cell control processing in which oxygen is pumped from around the second outer electrode to around the second inner electrode by controlling the second pump cell, thereby oxidizing hydrogen generated by the reduction of water in the first chamber in the second chamber;
a third pump cell control processing in which oxygen is pumped from around the third outer electrode to around the third inner electrode by controlling the third pump cell, thereby oxidizing carbon monoxide generated by the reduction of carbon dioxide in the first chamber in the third chamber; and
the control device derives the water concentration in the measurement gas based on the second pump current flowing through the second pump cell by the second pump cell control processing, while taking into account the third pump current flowing through the third pump cell by the third pump cell control processing.

2. The gas sensor according to claim 1,
wherein the control device derives the water concentration based on a corrected second pump current obtained by correcting the second pump current taking into account the third pump current, or derives the water concentration by correcting a provisional water concentration based on the second pump current taking into account the third pump current.

3. The gas sensor according to claim 2,
wherein the control device derives the corrected second pump current or the water concentration by applying a correction in such a manner that the greater the absolute value of the third pump current, the more the absolute value of the second pump current or the provisional water concentration tends to decrease.

4. The gas sensor according to claim 2,
wherein the control device derives a correction value based on the absolute value of the third pump current, and corrects the second pump current or the provisional water concentration using the correction value.

5. The gas sensor according to claim 4,
wherein the correction value is a subtraction value to the absolute value of the second pump current or the provisional water concentration, and
the control device corrects the second pump current or the provisional water concentration by subtracting the correction value from the absolute value of the second pump current or the provisional water concentration.

6. The gas sensor according to claim 5,
wherein the control device corrects the second pump current or the provisional water concentration using the same correction value derived based on the absolute value of the third pump current, regardless of the magnitude of the absolute value of the second pump current or the provisional water concentration.

7. The gas sensor according to claim 1,
wherein the control device derives a carbon dioxide concentration in the measurement gas based on the third pump current.

8. The gas sensor according to claim 1,
wherein at least two of the first outer electrode, the second outer electrode, and the third outer electrode are shared electrodes.

9. The gas sensor according to claim 1,
wherein the sensor element includes a reference electrode disposed inside the element body to contact a reference gas, and
the control device controls:
the first pump cell such that a first voltage between the reference electrode and the first inner electrode reaches a first voltage target value in the first pump cell control processing;
the second pump cell such that a second voltage between the reference electrode and the second inner electrode reaches a second voltage target value in the second pump cell control processing; and
the third pump cell such that a third voltage between the reference electrode and the third inner electrode reaches a third voltage target value in the third pump cell control processing.
